(19) United States
(12) Patent Application Publication
(10) Pub. No.: US 2024/0033273 A1
(43) Pub. Date: Feb. 1, 2024

(54) TOPICAL CANNABINOID COMPOSITIONS, ANTI-INFLAMMATORY CANNABINOID COMPOSITIONS, METHODS FOR FORMING, AND METHODS FOR TREATMENT

(71) Applicant: AGRONOMED MANAGEMENT, LLC, Exton, PA (US)

(72) Inventor: Jonathan COHN, Exton, PA (US)

(21) Appl. No.: 18/022,081

(22) PCT Filed: May 4, 2021
(86) PCT No.: PCT/US2021/030671

§ 371 Date: Feb. 17, 2023

Related U.S. Application Data
(60) Provisional application No. 63/025,663, filed on May 15, 2020.
(60) Provisional application No. 63/019,840, filed on May 4, 2020.

(51) Int. Cl. A61K 31/00; A61K 31/167

(52) U.S. Cl. CPC A61K 31/658; A61K 31/167

(57) ABSTRACT

A topical cannabinoid composition is disclosed, including a cannabinoid and a numbing agent. A method for making the topical cannabinoid composition is disclosed, including melting and combining butters and oils, then heating and adding waxes, then adding a numbing agent, and then adding at least one cannabinoid and, optionally, at least one terpene, to form the topical cannabinoid composition. A method for treating a skin condition is disclosed, including applying the topical cannabinoid composition to skin tissue having the skin condition. An anti-inflammatory cannabinoid composition is disclosed, including at least two but less than five species selected from the group consisting of cannabidiol, cannabidiolic acid, caryophyllene, pinene, and humulene. A method for reducing an inflammatory response associated with a viral infection is disclosed, including administering the anti-inflammatory cannabinoid composition to an individual experiencing the inflammatory response associated with the viral infection.

### RELATED APPLICATIONS

This application claims the benefit of and priority to U.S. Provisional Patent Application No. 63/019,840, filed May 4, 2020, entitled “Topical Use of Cannabinoids and Lidocaine,” and U.S. Provisional Patent Application No. 63/025,663, filed May 15, 2020, entitled “Novel Use of Cannabinoid Formulation for Coronavirus 2019,” which are hereby incorporated by reference in their entirety.

### FIELD OF THE INVENTION

Embodiments of the present invention relate to topical cannabinoid compositions combining cannabinoids and numbing agents, methods of making such compositions, and methods of treating skin conditions with such compositions, as well as anti-inflammatory cannabinoid compositions combining selected cannabinoids and/or cannabis-derived terpenes, and methods for reducing inflammatory responses associated with viral infections, such as, but not limited to, Coronavirus 2019 (“COVID-19”), comprising administering such anti-inflammatory cannabinoid compositions.

### BACKGROUND OF THE INVENTION

People suffer every summer from sunburn. Sunburn causes painful, dry, red skin and may even blister. Skin damage is caused by UVA and UVB rays from the sun that reach Earth's surface and penetrate skin.

Sunburn may be treated with anti-inflammatory drugs to relieve pain and discomfort, or by use of topical treatments such as Aloe Vera cream, moisturizers, ice packs, or lidocaine. Medication may negatively affect the liver if too much is taken and does not start to work until it is digested and released into the body. Creams, moisturizers, ice packs, and lidocaine will work to temporarily alleviate discomfort and some pain, but will wear off quickly and not aid in the healing process.

Chickenpox and Shingles are manifestations of the same virus, varicella zoster virus. A person having Shingles also had Chickenpox, also called varicella, as a child or adult. Chickenpox causes itchy blisters that start on the back, chest, or face and spread to the rest of the body. Once Chickenpox finishes its course, the virus retreats to the nerve tissue near the spinal cord and brain. Shingles is a rash that usually forms on just one side of the body and is accompanied with shooting pain. Shingles is called herpes zoster and starts when the virus reactivates traveling along the nerve fibers to the skin.

Chickenpox and Shingles may be treated with oral anti-viral medications to speed recovery, anti-inflammation drugs for swelling and pain, narcotic medications to reduce pain, antihistamines for itching, and/or anticonvulsants and antidepressants for prolonged pain. Oral medication may be harmful to the body as well as addicting like a narcotic. Topical numbing creams, gels, or patches may help with pain and capsaicin may aid in nerve pain after recovery. These topical aids may not last long, they do not aid in swelling and inflammation, and they cannot aid in healing and reduce scaring.

Tattoos are painful on the skin. They are a form of body modification where a design is made by inserting ink, dyes, and pigments into the dermis layer of skin. Redness, swelling, pain and bleeding are minor side effects of a tattoo while it is being placed on the body and for a few days following.

Tattooing is a painful body modification which may lead people to not want to get a tattoo or to stop the process not being able to sit to complete larger tattoos. If someone cannot sit through a tattoo or keep needing breaks, this may affect artist time and money. The process of the needles injecting ink into the skin causes sensations of burning and pain. Currently there are tattoo glides and after care products on the market that help numb the area while tattooing but none last long, and very few have cannabinoids in them to extend the use and help with pain and swelling.

Arthritis is a condition in which joints are inflamed. The main symptoms are joint pain and stiffness. Approximately 350 million people around the world suffer from this painful ailment with three types being osteoarthritis, rheumatoid arthritis, and psoriatic arthritis. Over time arthritis leads to the degeneration and destruction of the joints.

Arthritis treatments consist of non-steroidal anti-inflammatory drugs (“NSAIDs”) which may cause stomach problems irritating the lining of the stomach and cause bleeding. Disease-modifying anti-rheumatic drugs (“DMARDs”) are an alternative which may be taken orally or as an injection but may cause nausea, headaches, fatigue, and feeling wiped out or even more seriously liver damage. If DMARDs are not working by themselves, they may be paired with hydroxychloroquine which is an anti-malaria drug which may also cause eye damage. Steroids may be used to manage symptoms, but they may cause high blood pressure, weight gain, high blood sugar, and decrease bone health. Biologics have also been introduced, but they weaken the immune system, increasing the risk of infection. Some topical options such as corticosteroids and capsaicin creams are used in the treatment of arthritis to help reduce inflammation and joint pain, but they also suppress the immune system and do not last as long.

Hemorrhoid treatments include use of topical compositions, wipes, suppositories, and prescription medication. Topical creams may cause minor side effects such as irritation, dryness, rectal pain, burning, changes in menstrual periods, and increased facial or body hair growth, or more rare but serious side effects such as hives, difficulty breathing, swelling of lips, tongue, face, or throat, sever irritation, pain, or bleeding in or around rectum, sever stomach pain, seizures, sudden severe headache, blurred vision, anxiety, chest pain, irregular heartbeat, tremors, and ringing in the ears. Wipes have few reported side effects, but an allergic reaction is possible. Rectal suppositories may cause abnormal heart rhythm, chronic insomnia, dizziness, excessive sweating, fast heartbeat, head pain, involuntary quivering, loss of skin color, nervousness, rectal bleeding, severe irritation inside or around the rectum, and increased blood pressure. The most common side effects associated with prescription medications are blurred vision, eye pain, seeing halos around lights, mood changes, sleep problems, high blood sugar, irritability, fast heartbeat, depression anxiety, muscle weakness, increased urination, increased thirst, weight loss, hunger, numbness, and trouble breathing. Topical creams are used for pain and irritation, or to ease discomfort. Even if the topical composition contains lidocaine, the product will not last long, nor will it aid in healing and inflammation reduction.

Hemorrhoids, while rarely dangerous, may be very painful. Hemorrhoids are swollen veins in the lowest part of your rectum and anus. The walls of the blood vessels stretch so thin that the veins bulge and become irritated. Internal hemorrhoids are usually not very painful as they are far enough inside that they are remote from pain-sensing nerves. As such, bleeding will often be the only sign of internal hemorrhoids. External hemorrhoids are closer to pain-sensing nerves where they cause pain and bleeding.

COVID-19 is caused by the severe acute respiratory syndrome coronavirus-2 (SARS-CoV-2). Evidence on the pathophysiology of COVID-19 is rapidly emerging. However, it is yet to be understood why the underlying pathological mechanisms cause some patients severe illness while others experience only mild symptoms. Dependent on the unique patient, a respiratory tract infection caused by the virus may lead to inflammasome formation, resulting in secretion of proinflammatory cytokines. The patient's immune response determines the consequence and severity of this reaction.

COVID-19 lung pathology resembles that of SARS-CoV (the virus responsible for the previous SARS epidemic in 2002), causing respiratory tract infections and leading to the accumulation of anergic lymphocytes that express memory phenotype, acting as an important source of proinflammatory cytokines. An abundance of cytokine production may be caused by the antibody-dependent enhancement of the inflammatory reaction. The formation of immune complexes containing SARS-CoV-2 and non-neutralizing antibodies leads to a life-threating inflammatory response. These complexes bind to the Fc receptor for immunoglobulin, enabling SARS-CoV-2 to enter and replicate in new, non-pneumocyte, target cells. Symptoms and systems attacked by the virus. COVID-19 has been shown to cause coagulation and thrombosis in severe and fatal cases. The hypercoagulable state, potentially explained by the systemic inflammatory response, results in massive activation of coagulative pathways and at the same time, inhibition of fibrinolysis. It has been observed that up to 30% of patients with COVID-19 admitted into an intensive care unit (“ICU”) presented with symptoms of acute kidney injury (“AKI”). It has been reported that many patients with COVID-19 were diagnosed as having an acute myocardial injury, manifested by elevated levels of high-sensitive troponin.

Cannabinoids are believed to confer an anti-inflammatory effect in animals, for example, in the human body. In particular, orally ingested cannabinoids have been demonstrated to provide benefits in patients having conditions that involve inflammation. Ingestion of certain cannabinoids through nasal passage delivery has received FDA-approval for rare childhood epilepsy disorder(s). There is no known suggestion in the art that cannabinoids would be expected to positively affect the condition of individuals with viral infections.

### SUMMARY OF THE INVENTION

Cannabidiol is a non-psychoactive cannabinoid derived from a cannabis plant, in particular hemp. Cannabidiol may be extracted using various methods, including, but not limited to, ethanol extraction, CO₂ extraction, butane extraction, and cold pressing. Cannabidiol is becoming more widely used in pharmacology to help treat different ailments internally and externally in humans and other mammals. Cannabidiol is believed to be therapeutic and interacts with the endocannabinoid system. Cannabidiol binds to CB2 receptors found in various systems throughout the human body. Dermatologically, cannabidiol binds to CB2 receptors in the skin, creating homeostasis within an infected area. Homeostasis in this instance indicates that the cannabidiol is healing or repairing the area of skin which is in pain, burnt, cut, scarred, inflamed, etc. Research has shown that cannabidiol has antioxidant and anti-inflammatory properties. It inhibits the growth of bacteria and reduces swelling in muscles and joints. It has also shown to work as an anticonvulsant and anti-epileptic.

In one exemplary embodiment, a topical cannabinoid composition includes a cannabinoid and a numbing agent.

In another exemplary embodiment, a method for making a topical cannabinoid composition includes melting and combining butters and oils to form a butter-oil mixture, then heating and adding waxes to the butter-oil mixture to form a base mixture, then adding a numbing agent (such as, but not limited to, lidocaine) to the base mixture to form a numbing composition, and then adding at least one cannabinoid (such as, but not limited to, cannabidiol) and, optionally, at least one terpene, to the numbing composition to form the topical cannabinoid composition.

In another exemplary embodiment, a method for treating a skin condition includes applying a composition including a cannabinoid and a numbing agent to skin tissue having the skin condition.

In another exemplary embodiment, an anti-inflammatory cannabinoid composition includes at least two but less than five species selected from the group consisting of cannabidiol, cannabidiolic acid, caryophyllene, pinene, humulene, and combinations thereof.

In another exemplary embodiment, a method for reducing an inflammatory response associated with a viral infection includes administering an anti-inflammatory cannabinoid composition to an individual experiencing the inflammatory response associated with the viral infection, the anti-inflammatory cannabinoid composition including at least two but less than five species selected from the group consisting of cannabidiol, cannabidiolic acid, caryophyllene, pinene, and humulene.

Other features and advantages of the present invention will be apparent from the following more detailed description, taken in conjunction with the accompanying drawings which illustrate, by way of example, the principles of the invention.

### DETAILED DESCRIPTION OF THE INVENTION

In accordance with various embodiments, the present invention is directed to cannabinoid-based compositions, methods for making the same and methods for using the same. In comparison with composition not having one or more of the features described herein, embodiments of the present invention increase efficacy of treatment, duration of a treatment benefits, reduce inflammation, extend a period of relief from swelling, pain, or inflammation, accelerate a rate of healing of a wound or scarring, prevent infection by a virus such as a coronavirus, improve patient conditions to expedite the recovery process in patients testing positive for COVID-19, reduce patient time in intensive care, avoid or reduce use of ventilation equipment, increase quality of life during illness, avoid disease-related fatality, avoid disease-related morbidity, or combinations thereof.

In one embodiment, a topical cannabinoid composition includes a cannabinoid and a numbing agent. The topical cannabinoid composition may further include at least one of butter, oils, waxes, or terpenes. The topical cannabinoid composition may also, or alternatively, further include a pharmaceutically suitable carrier.

Suitable butters include, but are not limited to, coco butter, shea butter, or combinations thereof.

Suitable oils include, but are not limited to, coconut oil, medium-chain triglyceride (“MCT”) oil, jojoba oil, or combinations thereof.

Suitable waxes include, but are not limited to, beeswax, carnauba wax, candelilla wax, cetearyl alcohol, gyceryl stearate, Polysorbate 60, or combinations thereof.

Suitable terpenes include, but are not limited to, cannabis-derived terpenes such as caryophyllene, pinene, humulene, or combinations thereof. Terpenes may increase absorption of the cannabinoid, the numbing agent, or both.

Suitable numbing agents include, but are not limited to, lidocaine, bupivacaine, etracaine, benzocaine, prilocaine, or combinations thereof. In one embodiment, the topical cannabinoid composition includes lidocaine in combination with at least one of bupivacaine, etracaine, benzocaine, or prilocaine.

The cannabinoid may include any extract, such as an oil from any cannabis plant. In particular, cannabinoids may be obtained from hemp plants extracted using CO₂, hydrocarbons, dimethyl ether, and/or ethanol extraction processes. Generally, cannabinoid means and refers to one cannabinoid or a mixture of cannabinoids, cannabinoid isolates, or any other isolated and purified cannabinoid, terpene, or biomolecule product by the genus Cannabis plant, for example, cannabidiol. Cannabidiol (and other cannabinoids) are sourced from industrial hemp plants with THC levels less than 0.3%. Cannabinoids are refined and further processed to an isolate the pure result of chemical purification process. Cannabinoid isolates used may be about 98-99% pure.

Suitable cannabinoids include, but are not limited to, cannabidiol, cannabidiolic acid, cannabigerol, cannabigerolic acid, tetrahydrocannabinolic acid, cannabichromene, or combinations thereof. In one embodiment, the cannabinoid includes at least one of, but less than six of cannabidiol, cannabidiolic acid, cannabigerol, cannabigerolic acid, tetrahydrocannabinolic acid, and cannabichromene. In yet a further embodiment, the cannabinoid includes cannabidiol and at least one of, but less than five of, cannabidiolic acid, cannabigerol, cannabigerolic acid, tetrahydrocannabinolic acid, and cannabichromene. In an alternative embodiment, the cannabinoid consists essentially of cannabidiol, wherein “consists essentially of” includes trace impurities of other cannabinoids totaling less than 5% by weight of the amount of cannabidiol present. In a further alternative embodiment, the cannabinoid consists of cannabidiol.

In one embodiment, the topical cannabinoid composition includes both lidocaine as a numbing agent and cannabidiol as a cannabinoid.

The cannabinoid may be present in the topical composition in an amount relative to the total topical composition that is at least about 20 to about 20,000 ppm, alternatively about 20 to about 400 ppm, alternatively about 20 to about 1,000 ppm, alternatively about 1,000 to about ppm, alternatively about 5,000 to about 10,000 ppm, alternatively about 10,000 to about ppm, or any sub-range or combination thereof.

The numbing agent may be present in the topical composition in an amount relative to the total topical composition that is not more than about 400 ppm, alternatively at least about 1 ppm to about 100 ppm, alternatively at least about 50 ppm to about 150 ppm, alternatively at least about 100 ppm to about 200 ppm, alternatively at least about 150 ppm to about 250 ppm, alternatively at least about 200 ppm to about 300 ppm, alternatively at least about 250 ppm to about 350 ppm, alternatively at least about 300 ppm to about 400 ppm, alternatively about 40 pm to about 25,000 ppm, or any sub-range or combination thereof.

In one embodiment, wherein the cannabinoid includes cannabidiol and the numbing agent include lidocaine, the cannabidiol may be present in the topical composition in an amount relative to the total topical composition that is at least 20 ppm and the lidocaine may be present in the topical composition in an amount relative to the total topical composition that is not more than 400 ppm.

In one embodiment, a method for making a topical cannabinoid composition includes melting and combining butters and oils to form a butter-oil mixture, then heading and adding waxes to the butter-oil mixture to form a base mixture, then adding a numbing agent (such as, but not limited to, lidocaine) to the base mixture to form a numbing composition, and then adding a cannabinoid (such as, but not limited to, cannabidiol) and, optionally, terpenes, to the numbing composition to form the topical cannabinoid composition. In an alternative embodiment, the order of adding the cannabinoid and, optionally, terpenes and adding the numbing agent may be reversed, or the steps of adding the cannabinoid and, optionally, terpenes and adding the numbing agent may be combined so as to be performed simultaneously. The cannabinoid may be a full spectrum oil, an isolate, or both.

The topical cannabinoid composition may be used for topical application in the management of pain associated with any of a variety of issues including, but not limited to, arthritis, sunburn, chicken pox/shingles, hemorrhoids, tattooing, and similar conditions.

In one embodiment, a method for treating a skin condition includes applying the topical cannabinoid composition (including a cannabinoid and a numbing agent) to skin tissue having the skin condition. The skin condition may include, but is not limited to, swelling, pain, inflammation, a wound, scarring, or combinations thereof. Applying the composition to the skin tissue may extend a period of relief from the swelling, the pain, or the inflammation, or accelerates a rate of healing of the wound or the scarring, or combinations thereof, relative to an otherwise identical comparative composition lacking the cannabinoid.

Uses of the topical cannabinoid composition include topical application according to a treatment regimen as needed. In one embodiment, the cannabinoid is present at least in the amount of 93.75 mg, such as, in a representative embodiment, a composition is formulated to include about 375 mg cannabidiol per ounce, and about 163.93 mg numbing agent per ounce. In some embodiments, the composition may further comprise a terpene that is present in an amount that is about 1.31 mL (1.31 g) of 100% terpene blend per ounce of composition. In one example, a composition may be provided in a package that includes about 0.25 oz, having about 93.75 mg of cannabidiol total. In another example, the composition may be provided in a package that includes about 1 oz of composition, having about 375 g of cannabidiol. And in yet another example, the composition is provided in a package that includes about 2 oz, having about 750 mg of cannabidiol. Each such example includes amounts of numbing agent and optional terpenes as described herein. The compositions may modulate pain to enable prolonged pain relief that is improved significantly in comparison to topicals that lack cannabinoids.

Without being bound by any theory of mechanism, it is believed that when the topical compositions hereof are used as described, the effective duration of pain relief is extended when compared to a topical numbing agent without a cannabinoid. Thus, when compositions according to the disclosure are used, the effective half-life of lidocaine is extended. The numbing effects of lidocaine will be extended to help with pain and discomfort while the cannabinoid will also aid the tissue by reducing swelling and inflammation, help ease pain, and promote healing.

In some embodiments, the topical cannabinoid and lidocaine formulation promotes healing and aid to burnt skin. A superficial burn such as sunburn, which will heal quickly and may cause some skin peeling to full thickness burns which extend through the entirety of the dermal layers only healing itself by scarring or contracture. Lidocaine will numb the area reducing pain and sensitivity while the cannabinoid helps restore the skin cells, limiting peeling and scarring. Cannabinoids may help reduce pain in the damaged area of skin and promote healing of the skin.

In some embodiments, the topical cannabinoid and lidocaine formulation has anti-convulsant and numbing properties. Anti-convulsant medication may be used in nerve pain management with viral conditions such as chickenpox and shingles. Lidocaine may help numb the area aiding in pain and itch. Cannabinoids may promote the healing of the skin minimizing scarring and allow the lidocaine to be effective longer.

In some embodiment, the topical cannabinoid and lidocaine formulation's numbing properties will aid in the tattoo process. Lidocaine may numb the area to minimize pain while ink is being injected into the skin's dermis via penetration of needles. Lidocaine's numbing properties may aid in minimizing pain after the tattoo through aftercare application. Cannabinoids' anti-inflammatory properties may aid in decreasing skin inflammation throughout the tattoo process. Cannabinoids' skin regenerative properties may aid in the aftercare of the tattoo. Cannabinoids may extend the amount of time lidocaine is effective, extending the time an individual may tolerate and sit for tattoo administration.

In some embodiments, the topical cannabidiol and lidocaine formulation may aid in pain management for arthritis. Cannabinoids' anti-inflammatory properties may reduce inflammation in the joints relieving stiffness and pain. Lidocaine's numbing properties may relieve pain and soreness in the joints with the aid of cannabinoids to extend the time of effectiveness.

In one embodiment, an anti-inflammatory cannabinoid composition includes at least two but less than five species selected from the group consisting of cannabidiol, cannabidiolic acid, caryophyllene, pinene, humulene, and combinations thereof. In a further embodiment, the anti-inflammatory cannabinoid composition includes at least one cannabinoid selected from the group consisting of cannabidiol and cannabidiolic acid, and at least one cannabis-derived terpene selected from the group consisting of caryophyllene, pinene, and humulene. The anti-inflammatory cannabinoid composition may further include at least one of cannabigerol, cannabigerolic acid, tetrahydrocannabinolic acid, or cannabichromene.

The anti-inflammatory cannabinoid composition may be in any suitable form, such as an injectable solution, a suppository, a tincture for oral administration, or a powder having a particle size suitable for administration by nebulization.

In one embodiment, a method for reducing an inflammatory response associated with a viral infection includes administering the anti-inflammatory cannabinoid composition to an individual experiencing the inflammatory response associated with the viral infection. The viral infection may be any viral infection that induces an inflammatory response, including, but not limited to, COVID viruses such as COVID-19. The method for reducing an inflammatory response associated with a viral infection may further include the anti-inflammatory cannabinoid composition reducing viral load in the individual.

Modes of delivery may be selected by patient severity. In some embodiments, an oral administration is selected in the form of a tincture for patients showing moderate to mild symptoms. In other embodiments, particularly for more severe cases, nebulization is selected as the mode of administration. Nebulized forms include the formula to be made available in a suitable particulate form to achieve pulmonary absorption.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A topical cannabinoid composition, comprising:
a cannabinoid; and
a numbing agent.

2. The topical cannabinoid composition according to claim 1, further comprising:
at least one of butter, oils, waxes, or terpenes; and
a pharmaceutically suitable carrier.

3. The topical cannabinoid composition of claim 1, wherein the numbing agent includes an agent selected from the group consisting of lidocaine, bupivacaine, etracaine, benzocaine, prilocaine, and combinations thereof.

4. The topical cannabinoid composition of claim 3, wherein the numbing agent includes the lidocaine.

5. The topical cannabinoid composition of claim 4, further including at least one of the bupivacaine, the etracaine, the benzocaine, or the prilocaine.

6. The topical cannabinoid composition of claim 1, wherein the cannabinoid includes a species selected from the group consisting of cannabidiol, cannabidiolic acid, cannabigerol, cannabigerolic acid, tetrahydrocannabinolic acid, cannabichromene, and combinations thereof.

7. The topical cannabinoid composition of claim 1, wherein the cannabinoid includes cannabidiol and the numbing agent includes lidocaine.

8. The topical cannabinoid composition of claim 1, wherein the cannabinoid is present in an amount that is at least about 20 to about 20,000 ppm, and the numbing agent is present in an amount that is not more than about 400 ppm.

9. The topical composition of claim 8, wherein the cannabinoid includes cannabidiol present in an amount that is at least 20 ppm and the numbing agent includes lidocaine present in an amount that is not more than 400 ppm.

10. A method for making a topical cannabinoid composition, comprising:
melting and combining butters and oils to form a butter-oil mixture;
then heating and adding waxes to the butter-oil mixture to form a base mixture;
then adding lidocaine to the base mixture to form a numbing composition; and
then adding at least one cannabinoid and, optionally, at least one terpene to the numbing composition to form the topical cannabinoid composition.

11. The method of claim 10, wherein the at least one cannabinoid is a full spectrum oil, an isolate, or both.

12. A method for treating a skin condition, the method comprising:
applying a composition including a cannabinoid and a numbing agent to skin tissue having the skin condition.

13. The method of claim 12, wherein the skin condition includes swelling, pain, inflammation, a wound, scarring, or combinations thereof.

14. The method of claim 12, wherein applying the composition to the skin tissue extends a period of relief from the swelling, the pain, or the inflammation, or accelerates a rate of healing of the wound or the scarring, or combinations thereof, relative to an otherwise identical comparative composition lacking the cannabinoid.

15. An anti-inflammatory cannabinoid composition, comprising at least two but less than five species selected from the group consisting of cannabidiol, cannabidiolic acid, caryophyllene, pinene, humulene, and combinations thereof.

16. The anti-inflammatory cannabinoid composition of claim 15, wherein the anti-inflammatory cannabinoid composition includes at least one cannabinoid selected from the group consisting of cannabidiol and cannabidiolic acid, and at least one cannabis-derived terpene selected from the group consisting of caryophyllene, pinene, and humulene.

17. The anti-inflammatory cannabinoid composition of claim 15, further including at least one of cannabigerol, cannabigerolic acid, tetrahydrocannabinolic acid, or cannabichromene.

18. The anti-inflammatory cannabinoid composition of claim 15, wherein the anti-inflammatory cannabinoid composition is a tincture, an injectable solution, or a suppository.

19. The anti-inflammatory cannabinoid composition of claim 15, wherein the anti-inflammatory cannabinoid composition is a powder having a particle size suitable for administration by nebulization.

20. A method for reducing an inflammatory response associated with a viral infection, comprising administering an anti-inflammatory cannabinoid composition to an individual experiencing the inflammatory response associated with the viral infection, the anti-inflammatory cannabinoid composition including at least two but less than five species selected from the group consisting of cannabidiol, cannabidiolic acid, caryophyllene, pinene, and humulene.
